(19) United States
(12) Patent Application Publication
(10) Pub. No.: US 2020/0147169 A1
(43) Pub. Date: May 14, 2020

(54) COMPOSITION COMPRISING A HIGH CONCENTRATION OF ITURIN LIPOPEPTIDES

(71) Applicant: LIPOFABRIK, Villeneuve D'Ascq (FR)

(72) Inventors: François COUTTE, Aix (FR); Arnaud DELECROIX, Fleurbaix (FR); Abdelnasser EL GHAZOUANI, Lesquin (FR); Philippe JACQUES, Liege (FR)

(73) Assignee: LIPOFABRIK, Villeneuve D'Ascq (FR)

(21) Appl. No.: 16/632,061

(22) PCT Filed: Jul. 23, 2018
(86) PCT No.: PCT/FR2018/051883

§ 371 Date: Jan. 17, 2020

(30) Foreign Application Priority Data
Jul. 21, 2017 (FR) 17/56909

(51) Int. Cl. A61K 38/12; A61K 8/64; A01N 63/22; A23L 33/195; A23L 33/115

(52) U.S. Cl. CPC A61K 38/12; A61K 8/64; A01N 63/22; A23V 2002/00; A23L 33/115; A61K 2800/10; A23L 33/195

(57) ABSTRACT

The disclosure relates to compositions having a high concentration of iturinic lipopeptides. In particular, the compositions have an iturinic lipopeptide concentration of between 20 and 150 g/l; they are stable and homogeneous as a result of the presence of surfactants.

### CROSS-REFERENCE TO RELATED APPLICATIONS

This application is a National Phase Entry of International Patent Application No. PCT/FR2018/051883, filed on Jul. 23, 2018, which claims priority to French Patent Application No. 17/56909, filed on Jul. 21, 2017, both of which are incorporated by reference herein.

### BACKGROUND AND SUMMARY

The invention relates to compositions with a high concentration of iturinic lipopeptides. In particular, the compositions according to the invention have a concentration of iturinic lipopeptides between 5 and 200 g/L, and are stable and homogeneous thanks to the presence of surfactants and/or hydrotropic molecules.

Iturinic lipopeptides are molecules produced by different strains of Bacillus sp. and more particularly strains of Bacillus subtilis, Bacillus amyloliquefaciens, Bacillus megaterium, Bacillus thermoamylovorans, Bacillus thermocloacae, Bacillus firmus, Bacillus mojavensis, Bacillus velenzensis, Bacillus valissmortis. Non-exhaustive examples include Bacillus subtilis strains ATCC6633, W23, ATCC19659, DSM23117, QST713 or AQ713, FMBJ, 3-10, RB14, BH072 and their derivatives, Bacillus amyloliquefaciens strains FZB42, KB3, SYBC H47, GA1 and their derivatives.

This family of molecules includes iturin A, AL and C, mojavensin, mycosubtillin and bacillomycins A, B, C, C, D, F, L and Lc. These components are known mainly for their antifungal properties but they also have antibacterial properties. These properties come from their amphiphilic nature, which allows them to interact with different membrane components.

Iturinic molecules can be produced in solution in the fermentation broth by the different strains mentioned above, then they can be recovered according to methods known to the skilled person in order to extract, concentrate and purify them from a culture supernatant. Nevertheless, their behaviour in solution is dependent on their concentration. Indeed, it is known that above the critical micellar concentration (between 10 and 20 mg/L), iturinic compounds like other lipopeptide molecules form more or less complex micelles. In addition, the size of these micelles will evolve to form increasingly complex structures as their concentration increases from an average size of 10 nm to concentrations belowl 500 mg/L (Jauregi et al., 2013), to vesicular structures with an average diameter of 150 nm above 1 g/L (Grau et al. 2001), and then to lamellar two-layer superstructures at a concentration of 10 g/L (Hamley et al., 2013). As disclosed by these studies on mycosubtilin (Hamley et al., 2013) or iturine A (Grau et al. 2001), these superstructures are not found in other lipopeptide molecules of the surfactin family for example. In addition, the increase in the concentration of iturinic compounds tends to increase their interaction with the proteins contained in the culture medium and thus to increase the size of the structures leading to their insolubility (Jauregi et al. 2013). These physico-chemical properties of iturinic compounds therefore raise solubility problems, resulting in unstable compositions that tend to precipitate or gel when the concentration of iturinic lipopeptides in the compositions is increased.

The prior art refers to few compositions with a concentration of iturinic lipopeptides above 5 g/L and even fewer with a concentration above 20 g/L, mainly because it is known to the skilled person that iturinic lipopeptides tend to form high concentration micelles, making the compositions unstable because of iturin micelles preventing their good solubility. However, in the prior art, Japanese patent 3P2003 128512 (SHOWA DENKO) is known to contain cosmetic compositions, including surfactants, to which iturin and surfactin are added for their antimicrobial properties, but the stability and homogeneity of iturin in these compositions are not mentioned, since they are not the main compound of the various compositions.

Also known is the US patent Publication No. 2016/183537 which describes a composition from Bacillus amyloliquefaciens containing iturin, surfactin and fengycin to improve plant growth or protect plants. This document does not mention the concentrations of the molecules contained in this composition. No mention is made of high iturine concentrations.

The publication of Choukri HBID et al. describing the influence of lipopeptide extracts on oxygen transfer during fermentation is also known. These extracts correspond to a mixture of iturine/surfactin at concentrations up to 4 g/L. The possibility of a higher iturin concentration is not described in the document. Thus, none of the prior art documents encourages the skilled person to make compositions with a high concentration of iturinic lipopeptides, because this person knows that they will not be stable and homogeneous and therefore have few applications.

The inventors have shown that it is possible to prepare a stable and homogeneous composition in which the concentration of iturinic lipopeptides is above 5 g/L by adding molecules with surfactant properties. Indeed, the inventors have shown that surfactants improve the solubilization of iturinic lipopeptides beyond 5 g/L, in particular at 10 g/L and 20 g/L and up to 50, 100 or even 200 g/L without the appearance of a precipitate or a gel. The present invention has applications in the production of concentrated solutions of antifungal, antibacterial or biosurfactant biopesticide molecules for the plant protection industry, but also in the food, cosmetics, pharmaceutical and petroleum fields.

### BRIEF DESCRIPTION OF THE DRAWINGS

FIG. 1 is a graph showing solubility of iturinic lipopeptides as a function of their concentration;

FIG. 2 is a graph showing solubility of different compositions including iturinic lipopeptide;

FIG. 3 includes graphs showing minimum viscosity of different concentrated iturinic compositions; and

FIG. 4 includes graphs showing shear stress at maximum viscosity of different concentrated lipopeptide compositions.

### DETAILED DESCRIPTION

The general concept of the invention relates to a stable and homogeneous liquid composition comprising a mixture of lipopeptides in which the concentration of iturinic lipopeptides is above 5 g/L. In such a composition, lipopeptides are combined with molecules with surfactant properties, whereby the concentrated iturinic lipopeptide composition remains stable and homogeneous. A first object of the invention relates to a stable and homogeneous composition comprising a concentration of iturinic lipopeptides above 20 g/L and one or more surfactant(s) chosen from the families of anionic surfactants, non-ionic surfactants or oils.

“Stable and homogeneous” composition means a composition that does not include precipitate, and remains homogeneous at temperatures between 4° C. and 40° C. The absence of caking is also an important criterion, i.e. the solution is liquid and not in a gel form. The term liquid should be understood in the sense of the invention as a fluid composition, i.e. a composition with a low viscosity (the shear stress to shear velocity ratio).

The term homogeneous should be understood as a mixture in which the different components cannot be distinguished with the naked eye after stirring. Homogeneity can be assessed by the uniformity of concentrations at any point in the mixture and the absence of structures or micro-structures (caking or precipitate) visible to the eye. Such homogeneity of the mixture is advantageous because it allows uniform concentrations of iturinic lipopeptides, which can then be diluted in an aqueous solution.

The term stable should be understood as the fact that the composition remains homogeneous over time. Thus, a stable and homogeneous mixture is a mixture with a uniform concentration that remains constant over time.

According to a preferred embodiment, the composition according to the invention includes between 20 and 200 g/L of iturinic lipopeptides. It may include, for example, 20, 25, 30, 40, 50, 60, 70, 80, 90, 100 or 200 g/L of iturinic lipopeptides. Preferably the concentration of iturinic lipopeptides in the composition is between 20 and 150 g/L, or between 20 and 100 g/L. Even more preferably, the concentration of iturinic lipopeptides in the composition is above 50 g/L, i.e. between 50 and 150 g/L, or between 50 and 100 g/L or between 50 and 80 g/L.

“Molecules of the iturins family” means iturin A, mojavensin, mycosubtilin, and bacillomycins A, B, C, D, F and L. The compositions of this invention include molecules with surfactant properties. Molecules with surfactant properties”, also known as “surface-active agents” or simply “surfactants”, include amphiphilic molecules, surfactants, lipopeptides, such as surfactin, fengycin (or plipastatin), chemical surfactants and biological surfactants, such as rhamnolipids, polysaccharides, etc. . . . These molecules can be used alone or in combination in the compositions of the invention.

Surfactants include heparin, hyaluronic acid, dextran, amylose, chitosan, anionic surfactants derived from amino acids, non-ionic surfactants derived from poly-glycosides, hydrotropes surfactants, lipopeptides such as surfactant isomers and/or fengycin isomers (or plipastatin), rhamnolipids and vegetable oils. More specifically, the invention relates to anionic, non-ionic and oil surfactants. Surfactants of the anionic surfactants family are selected from: surfactins, fengycins, or amino acid derivatives.

“Molecules of the surfactins family” means surfactins A, B, C, lichenysin and pumilacidin. “Molecules of the fengycins family” means fengycins A and B, plipastatins A and B and agrastatin. Surfactants of the non-ionic surfactants family are selected from: fatty alcohol axalkylate, pentylene glycol and its derivatives, hydrotropic molecules of the alkylpolyglycoside type (alkypolyglycoside and alkylthoxypolyglycoside), textured polyglycoside molecules (xanthan gum, arabic gum, tragacanth gum, guar gum, locust bean gum, tamarind gum, pectin, gel gum, carrageenates, agar-agar, alginates.) The surfactants of the oil family are chosen from: oils and extracts of modified (acidified, methylated, esterified) oils in particular from: almond, groundnut, argan, avocado, rapeseed, lorenzo, neem, hazelnut, cashew nut, macadamia nut, olive, pistachio, rice, oleic sunflower, camelina, flax, borage, safflower, hemp, cotton, wheat germ, but, nut, carnation, evening primrose, barley, pumpkin seeds, grape seeds, peas, sesame, soya, sunflower.

Iturinic lipopeptides will be micro-emulsified in these oily compounds or fats to obtain the surfactant effect of modified oils and oil extracts. The compositions of the invention may also contain additives such as lipid sources, salts and solvents. These additives can participate in solubilization without being surfactants. Examples of solvents include ethanol, methanol, acetonitrile, dimethyl sulfoxide, butanol, pentanol, acetone.

According to a first aspect, the invention relates to a composition comprising between 5 and 50 g/L of iturinic lipopeptides and between 1 and 40 g/L of surfactin. In a particular composition, iturinic lipopeptides are mycosubtilins. In another particular composition, the amount of iturinic lipopeptides is preferably between 10 g/L and 50 g/L, preferably between 20 and 50 g/L, and even more preferably between 30 and 50 g/L.

According to a second aspect, the invention relates to a composition comprising between 5 and 100 g/L of iturinic lipopeptides, between 1 and 40 g/L of surfactin and between 1 and 100 g/L of fengycin (or plipastatin). In a particular composition, the amount of iturinic lipopeptides is preferably between 10 and 80 g/L, preferably between 20 and 80 g/L, most preferably between 30 and 80 g/L.

According to a third aspect, the invention relates to a composition comprising between 5 and 100 g/L of iturinic lipopeptides, between 1 and 40 g/L of surfactin and between 1 and 30 g/L of chemical surfactants. In a particular composition, the amount of iturinic lipopeptides is preferably between 10 and 80 g/L, preferably between 20 and 80 g/L, preferably between 30 and 80 g/L. In a particular embodiment, chemical surfactants may be non-ionic surfactants of the polyglycoside family or anionic surfactants derived from amino acids. Preferably, chemical surfactants consist of a mixture of anionic surfactants and non-ionic surfactants.

According to a fourth aspect, the invention relates to a composition comprising between 5 and 100 L of iturinic lipopeptides, between 1 and 40 g/L of surfactin, between 1 and 100 g/L of fengycin (or plipastatin) and between 1 and 30 g/L of chemical surfactants. In a particular composition, the amount of iturinic lipopeptides is preferably between 1 and 80 g/L, preferably beween 20 and 80 g/L, preferably between 30 and 80 g/L.

According to a fifth aspect, the invention relates to a composition comprising between 5 and 10 g/L of micro-emulsified iturinic lipopeptides in oily or fat compounds, such as vegetable oil. In such a composition, the oil concentration is between 1% and 100%, preferably between 50% and 100%, particularly preferably between 80% and 100%. Among the vegetable oils that can be used are corn oil, but also modified almond, peanut, argan, avocado, rapeseed, lorenzo, neem, hazelnut, cashew nut oils and extracts, macadamia nuts, olive, pistachio, rice, oleic sunflower, camelina, flax, borage, safflower, hemp, cotton, wheat germ, but, nuts, carnation, evening primrose, barley, pumpkin seeds, grape seeds, peas, sesame, soya, and sunflower.

Preferably the oils used are from groundnuts, olives, oleic sunflowers, flax, corn, nuts, soybeans and sunflowers. Such oils have the advantage of being available in large quantities and at low cost. Even more preferably, the oil used is from corn or sunflower peanuts. In a preferred embodiment, such a composition includes between 10 and 80 g/L, preferably between 20 and 80 g/L of mycosubtilin, preferably between 30 and 80 g/L and quite preferably between 50 and 80 g/L of mycosubtilin.

Thus, a particular composition according to the invention includes:

2 to 8% iturinic lipopeptides

3% chemical surfactants consisting of a mixture of non-ionic and anionic surfactants, each 1.5%

2 to 4% surfactants

0 to 10% fengycins or plipastatins.

In a particular embodiment, the composition comprises about 3% lipopeptides (1.25% mycosubtilin and 1.85% surfactin), 1.5% of a non-ionic surfactant derived from poly-glycosides and 1.5% of an anionic surfactant derived from amino acids. In another particular embodiment, the composition includes about 5% lipopeptides (2.45% mycosubtilin and 2.65% surfactin) and 3 to 8% fengycin (or plipastatin). In another particular embodiment, the composition includes between 30 and 80 g/L of mycosubtilin and 30 to 80 g/L of fengycin (or plipastatin).

In a particular embodiment the composition includes a concentration of iturinic lipopeptides between 20 and 100 g/L and the surfactants are an extract of vegetable oil, polyglycoside derivatives and surfactin. In another particular embodiment the composition includes a concentration of iturinic lipopeptides between 20 and 100 g/l and the surfactants are an extract of methylated vegetable seed oil and polyglycerol coconut oil ester (Synergen OS™). In another particular embodiment the composition includes a concentration of iturinic lipopeptides between 20 and 100 g/L and the surfactants are a mixture of alkylpolyglucoside (Simulsol™), amino acid derivative (Proteol™) and surfactin.

In another particular embodiment the composition includes a concentration of iturinic lipopeptides between 20 and 100 g/L and the surfactants are a mixture of fatty alcohol oxalkylate (Emulsogen™) and surfactin. In another particular embodiment the composition includes a concentration of iturinic lipopeptides between 20 and 100 g/L and the surfactants are a mixture of vegetable oil, xanthan gum and surfactant. The compositions according to the invention can be ready for use or in concentrated form; the concentrated solutions can be diluted before use.

A second object according to the invention relates to a dehydrated composition comprising a mixture of lipopeptides in which the concentration of iturinic lipopeptides is above 0.5%. In a preferred embodiment, the concentration of iturinic lipopeptides is above 2%. Such dehydrated compositions are in powder or lyophilized form and are “ready to use”; indeed, it is sufficient to rehydrate them in 100 mL of solution to obtain a composition whose iturinic lipopeptide concentration is at least 5 g/L. In a preferred embodiment, such compositions have a concentration of iturinic lipopeptides of at least 20 g/L.

The ingredients and their relative quantities in these solid compositions are the same as those described above for liquid compositions. Indeed, the concentrations by dry weight of the various components of these dehydrated solutions make it possible to obtain liquid compositions whose iturinic lipopeptide concentration is above 5 g/L which are stable and homogeneous according to the invention. In a preferred embodiment, the concentration of iturinic lipopeptides is above 20 g/L.

A third object of the invention relates to a method for preparing a composition comprising a mixture of lipopeptides in which the concentration of iturinic lipopeptides is above 5 g/L. In a preferred embodiment, the concentration of iturinic lipopeptides in the mixture is 20 g/L. The iturinic lipopeptides of the composition can be obtained by fermentation of a strain of Bacillus sp. and harvested and concentrated from the culture supernatant.

The compositions according to the invention can be prepared using commercially available lipopeptide preparations in powder form. These starting powders contain a variable amount of iturinic lipopeptides ranging from 10 to more than 80%, for example 15% or 75%. These powders may contain a mixture of mycosubtilin and surfactin in a ratio of 30/70 to 70/30 or even 95/5, for example 35/65, 40/60, 45/55 or 50/50.

To prepare a composition according to the invention, at least one surfactant must be added to a solution containing a high content of iturinic lipopeptides. The method for preparing such compositions may include an additional dehydration step in order to provide a “ready to use” composition in powder form, the characteristics of which are in accordance with the compositions of this invention once rehydrated. Thus the method makes it possible to prepare liquid or solid solutions (in powder or lyophilisate form). Such concentrated solutions are particularly suitable for storage and distribution. They must be diluted before use. Therefore, a composition according to the invention can be obtained by dissolving a powder allowing the reconstitution of a liquid composition as defined above.

Compositions according to the invention have applications in the fields of food, plant protection and cosmetics as well as in the medical and pharmaceutical fields. The invention is illustrated with the following examples.

### EXAMPLES

### A. Preparation of Solutions Containing Lipopeptides From Supernatant of Bacillus Strain Cultures

1—Obtaining Culture Supernatants

Lipopeptides are obtained from an aerobic fermentation process of a Bacillus strain derived from Bacillus subtilis strain ATCC 6633 for the production of iturinic lipopeptides and strains derived from Bacillus subtilis strain 168 for the production of surfactin and/or fengycin (or plipastatin). The culture is carried out in a stirred medium containing a carbon source (glucose, sucrose, . . . ), a nitrogen source (ammonia sulphate, peptone . . . ) and trace elements at 30° C. The pH is maintained at a value of 7. The culture is harvested after 48 to 72 hours. It is then centrifuged or filtered to remove the cells. The culture supernatant is then concentrated.

2—Preparation of Compositions with a High Content of Iturinic Lipopeptides From Culture Supernatants

To obtain compositions with a high content of iturinic lipopeptides, culture supernatants can be concentrated. The concentration of the culture supernatant can be obtained by any method known to the skilled person, in particular:

By tangential ultrafiltration using a membrane with a cut-off threshold of 1 KDa to 300 KDa. For example, 1000 L of culture supernatant obtained as described above are concentrated by passing over the membrane to obtain a retentate of a volume of 10 to 100 L. The small molecules are then removed by one or more diafiltration steps. The solution thus obtained can be dried by lyophilization, atomization or used as it is.

By precipitation at acidic pH: a decrease in pH in order to selectively precipitate lipopeptides is achieved. A addition of concentrated sulphuric acid is added to the supernatant obtained. After obtaining a final pH around 1, the solution is left to be stirred for 2 to 12 hours. A centrifugation allows to recover a cull of material containing the lipopeptides. This cull is then dissolved by adding water and soda to obtain a pH value between 7 and 8.5. The solution thus obtained can be dried by lyophilization, atomization or used as it is.

By evaporation: the supernatant is concentrated by vacuum evaporation. For example, 20 L are introduced into a rotavapor and concentrated at 1 to 2 L.

The percentage of lipopeptides at the end of one of these two examples of preparation is between 0.5 and 20% (weight/volume).

### B—Preparation of Iturinic Lipopeptide Concentrated Compositions

1—Study of the Effect of Different Surfactants on the Solubility of an Iturinic Lipopeptide and in a High Concentration Composition

An iturinic lipopeptide (here mycosubtilin), having a purity (on dry matter) of between 45% and 80%, was solubilized alone in water at different concentrations and this control composition was compared to different compositions to which different surfactant compounds were added as described in the application. After 15 days of storage at 21° C., only compositions with a rheological behaviour of the fluid type and a homogeneous appearance (without flow preventing caking and/or phase shift and/or precipitation) are selected, mixed by stirring, then these compositions are centrifuged for 10 minutes at 10,000 g and the supernatant is analysed using the RP-HPLC reference method. This method makes it possible to check the solubility of the iturinic compounds that are then found in the supernatant after centrifugation.

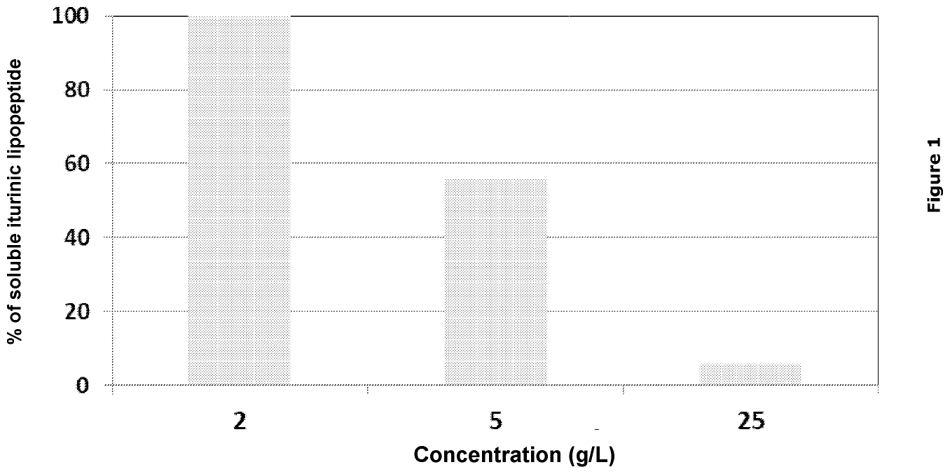

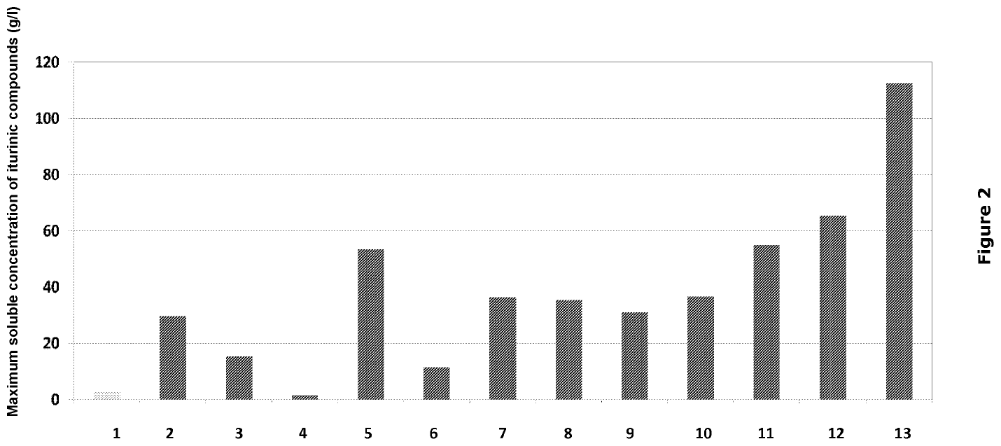

The solubility of iturinic lipopeptide (here mycosubtilin) alone in an aqueous solution as a function of its concentration is shown in FIG. 1. The maximum concentration of iturinic lipopeptides soluble in the different compositions is shown in FIG. 2. No surfactant is added to composition 1 which contains only iturinic lipopeptides.

The surfactants added in compositions 2 to 13 are:

10% fengycins (composition 2)

alkylpolyglucosides and amino acid derivatives (Simulsol™/Protéol™) at a rate of 10% each (composition 3)

soya lecithin (4%) and poly-glycoside derivatives (0.1%) (Elvis™/Xanthan Gum) (composition 4)

methylated vegetable seed oil extract and polyglycerol ester from coconut oil (0.2%) (Synergen OS™) (composition 5)

non-ionic surfactant of fatty alcohol oxalkylate type (0.2%) (Emulsogen™) (composition 6)

non-ionic surfactant of the pentylene glycol type (50%) (composition 7)

0.4% poly-glycoside derivatives (AlkylEthoxyGlycoside=AEG) (composition 8)

4% surfactins (composition 9)

4% surfactins and 10% fengycins (composition 10)

vegetable oil extract (30%) and derivatives of poly-glycosides (0.1%) and surfactin (4%) (Corn oil/Xanthan Gum/Surfactin) (composition 11)

alkylpolyglucoside and amino acid derivatives (Simulsol™/Protéol™) at a rate of 10% each and 4% surfactin (composition 12)

non-ionic surfactant of 0.2% fatty alcohol oxalkylate type (Emulsogen™) and 4% surfactin (composition 13).

Analysis of the Results:

FIG. 1 shows the solubility of iturinic lipopeptides (here mycosubtilin) in an aqueous solution at room temperature as a function of their concentration. In FIG. 1, it can be observed that mycosubtilin alone solubilizes very well in an aqueous solution at a maximum concentration of 2 g/L, but beyond this concentration (e.g. between 5 and 25 g/L) a precipitate that settles can be observed and only a small part remains soluble. FIG. 2 shows the solubility analysis of different compositions including an iturinic lipopeptide (here mycosubtilin) and different compounds to obtain high and soluble concentrations of iturinic lipopeptide after 15 days of storage at room temperature. The measurement is made on the supernatant after a centrifugation step to eliminate insoluble substances.

In FIG. 2, it can be observed that the addition of different surfactants significantly increases the solubility of mycosubtilin, particularly with anionic compounds alone or in combination with non-ionic surfactants. The presence of fengycin or surfactin allows to obtain a solubility of mycosubtilin close to 30 g/L. Surprisingly and in an original way, the combination of these two anionic lipopeptide compounds makes it possible to achieve an even higher iturinic compound solubility. It should also be noted that the use of certain surfactants despite a homogeneous and stable composition does not result in solubilization above 20 g/L. This is the case of soy lecithin coupled with Xanthan gum (max=1.5 g/L), the case of the non-ionic compound Emulsogen™ (Clariant) (max=11.6 g/L) but also the non-ionic compounds Simulsol™ and anionic Protéol™ (Ceppic) (max=15.3 g/L). The presence of a significant precipitate after the centrifugation step was noted for these compositions. On the other hand, when these compositions are supplemented by another anionic lipopeptide compound such as surfactin, the solubility of mycosubtilin increases to values between 55 and 120 g/L. Other non-ionic compounds such as penthylene glycol and alkylthoxyglycoside also provide solubility above 30 g/L. The composition allowing maximum solubility of the iturinic compound (112 g/L) is the one containing an anionic lipopeptide (surfactin) and a non-ionic surfactant (Emulsogen™).

2—Preparation of Compositions with a High Iturinic Lipopeptide Content From Lipopeptide Powders

A composition with a high content of iturinic lipopeptides can be prepared from lipopeptide powder, as illustrated in the following two examples of compositions:

Composition 1: A composition comprising about 3.1% (m/v) lipopeptides (1.25% mycosubtilin and 1.85% surfactin) was obtained by re-slurrying a lipopeptide powder comprising a 40/60 ratio of a mycosubtilin/surfactin mixture and a purity of about 15% in aqueous phase (pH between 7.5 and 8.5).

Composition 2: A composition comprising about 5.1% (m/v) lipopeptides (2.45% mycosubtilin and 2.65% surfactin) was obtained by re-slurrying a lipopeptide powder comprising a 45/55 ratio of a mycosubtilin/surfactin mixture and a purity of about 75% in aqueous phase (pH between 7.5 and 8.5).

3—Preparation of Compositions with a High Content of Iturinic Lipopeptides Comprising Surfactants.

Surfactants have been added to compositions 1 and 2 described above to evaluate the effect of such molecules on the properties of the compositions.

Composition 1A: Two surfactants were added to composition 1 each in a proportion of 10% (v/v), namely a non-ionic hydrotropic surfactant derived from poly-glycosides (Trade name=Simulsol′″ SL 7C marketed by Seppic) and an anionic surfactant derived from amino acids (Trade name=Proteol′″ APL marketed by Seppic).

Composition 2A: 3 to 8% (w/v) fengycin (or plipastatin) was added to composition 2.

4—Characterization of the Obtained Compositions: Homogeneity and Stability Over Time.

a. Stability Test at Room Temperature:

The appearance of the compositions with and without surfactants was studied at room temperature, here at about 21° C.

Composition 1: The addition of the two surfactants made it possible to obtain a homogeneous composition (composition 1A) and to avoid any precipitation or sedimentation phenomenon. The composition 1A containing surfactants has a cloudy but homogeneous appearance.

Composition 2: The addition of fengycin (or plipastatin) has made it possible to obtain a homogeneous composition and avoid any phenomenon of caking (which occurs in the absence of fengycin or plipastatin). The composition 2A with fengycin (or plipastatin) has a clear and homogeneous appearance.

b. Stability Tests at Low Temperature:

Concentrated lipopeptide solutions (Compositions 1, 2 and 1A, 2A) were placed at 4° C. for a period of 1 to 30 days. Only solutions supplemented with surfactants or fengycin (or plipastatin) (i.e. 1A and 2A) remained homogeneous and no deposition was observed. Without these additions, concentrated lipopeptide solutions are likely to cake or cause a significant insoluble deposit.

5—Characterization of the Rheology of the Compositions Obtained: Impact of Storage at Room Temperature and Accelerated Ageing

a. Methods of Analysis:

The 2 compositions described above 1 and 2 and those containing additives 1A and 2A were stored for 15 days at 21° C. and 15 days at 54° C. (to simulate accelerated ageing). The storage of compositions at high temperature (54° C.) is a method known to the skilled person, which makes it possible to mimic an accelerated ageing of the compositions by increasing the Brownian movement, accelerating the destabilisation of the compositions.

At the end of these periods, the rheology of the compositions was studied using a compact Anton PAAR MCR102 modular rheometer. The method consisted in monitoring the shear velocity when the shear stress gradually increased between 1 and 100 Pa (test duration: 500 s). Viscosity (the shear stress/shear velocity ratio) was also expressed as a function of shear stress. This made it possible to visualize the evolution of the viscosity during the test. These tests were performed on triplicate samples.

Two rheological parameters were studied on these compositions after storage at 21° C. or 54° C., namely:

The minimum viscosity (Pa·S)

Shear stress at maximum viscosity (Pa).

b. Results:

All samples showed non-Newtonian fluid behaviors (viscosity was not constant during the tests). As the shear stress increased, the viscosity decreased to close to a minimum viscosity. The decrease in viscosity under stirring can be explained by a gradual alignment of the structural units (or molecules) in the direction of the flow as the shear velocity increases, thus promoting the flow of the different liquid layers. The fluids are then referred to as shear-thinners and can be characterized by their minimum viscosities (in the case of the tests applied, this was the viscosity at the end of the test) and by the shear stress at the maximum viscosity (Pa).

Study of the Minimum Viscosity:

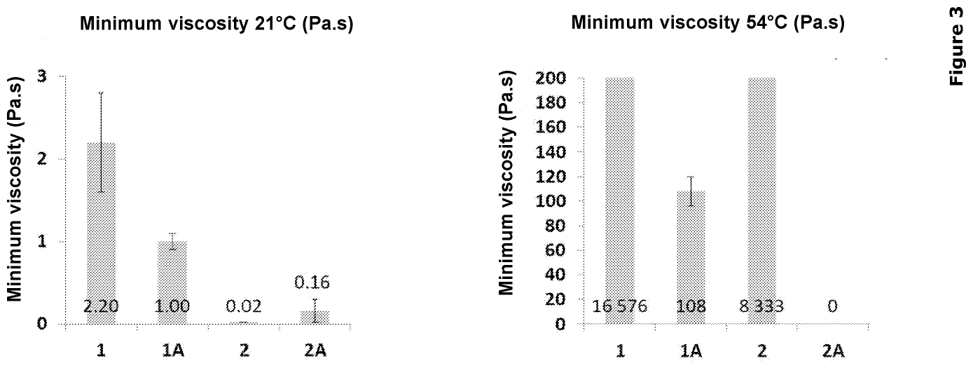

FIG. 3 shows the analysis of the minimum viscosity of the different concentrated iturinic compositions with or without the addition of surfactants and other additives A, after storage at 21° C. or 54° C. The results presented in FIG. 3 show that the minimum viscosity of composition 1A after storage at 21° C. is significantly different and lower than that of the unformulated composition 1. No significant difference is observed between compositions 2 and 2A after storage at 21° C. Nevertheless, in the ageing test at 54° C., the minimum viscosity of composition 2 very significantly increased while that of composition 2A remained close to 0. This shows the very important effect of adding fengycin (plipastatin) to the latter to facilitate the solubility of iturinic lipopeptides. A similar effect can be observed between composition 1 and 1A after storage at 54° C., showing the interest of the formulation of composition 1A.

Study of Shear Stress at Maximum Viscosity:

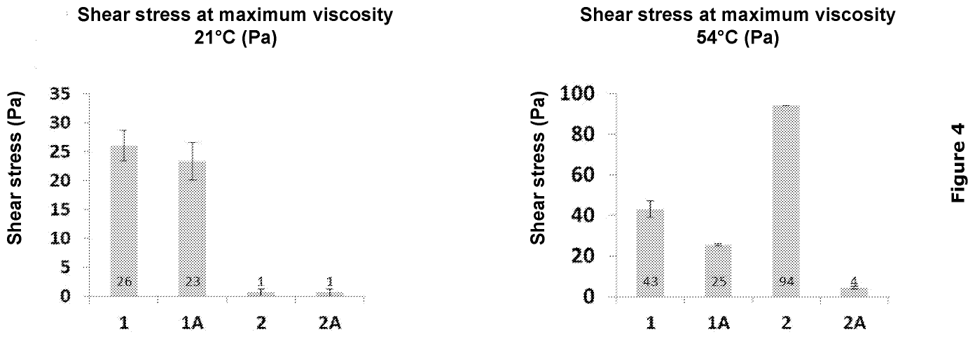

FIG. 4 shows the analysis of shear stress at the maximum viscosity of the different concentrated lipopeptide compositions with or without the addition of surfactants and other additives A, after storage at 21° C. or 54° C. The results presented in FIG. 4 show that the shear stresses at the maximum viscosity after storage for 15 days at 21° C. of composition 1A are lower than those obtained with the unformulated composition 1. No significant difference was observed between compositions 2 and 2A. Nevertheless, when studying the impact of accelerated ageing at 54° C. on this parameter, the results in FIG. 4 show that the shear stress very significantly increases in non-formulated compositions 1 and 2, while those of the formulated compositions 1A and 2A remained almost identical to those obtained at 21° C. These results show the very positive impact of formulations according to the invention to facilitate the solubilization over time with a high concentration of iturinic compounds.

As a conclusion of these studies, it can be established that:

Composition 1A makes it possible to solubilize iturinic compounds while avoiding precipitate and/or gel formation and this is characterized by a significant decrease in the minimum viscosity and shear stress at maximum viscosity either after storage at 21° C. or even more significantly during ageing thereof. Compositions 1 and 1A are related to non-Newtonian Casson-type fluids.

The composition 2A allows iturinic compounds to solubilize, while avoiding the formation of precipitate and/or gel after a long storage period and this is characterized by a significant decrease in the minimum viscosity and shear stress at the maximum viscosity after storage at 54° C. Compositions 2 and 2A are related to non-Newtonian fluids of the shear-thinning type at first, but the unformulated composition 2 becomes very thick during ageing thereof.

1. A stable and homogeneous composition, comprising a concentration of iturinic lipopeptides above 20 g/L, and one or more surfactant(s) chosen from the families of: anionic surfactants, non-ionic surfactants or oils.

2. A composition according to claim 1, wherein the concentration of iturinic lipopeptides is between 20 and 150 g/L.

3. A composition according to claim 1, wherein the surfactants of the anionic surfactant family are chosen from: molecules of the surfactin family, molecules of the fengycin family, or amino-acid derivatives.

4. A composition according to claim 1, wherein the surfactants of the non-ionic surfactant family are chosen from: fatty alcohol axalkylate; pentylene glycol and its derivatives; hydrotropic molecules of the alkylpolyglycoside type including: alkypolyglycoside and alkylethoxypolyglycoside; or molecules of the polyglycoside texture agent type including: xanthan gum, gum arabic, tragacanth gum, guar gum, locust bean gum, tamarind gum, pectin, gellan gum, carrageenates, agar-agar, or alginates.

5. A composition according to claim 1, wherein the surfactants of the oil family are chosen from: oils and extracts of modified including acidified, methylated, or esterified oils including at least one of: almond, groundnut, argan, avocado, rapeseed, lorenzo, neem, hazelnut, cashew nuts, macadamia nuts, olive, pistachio, rice, oleic sunflower, camelina, flax, borage, safflower, hemp, cotton, wheat germ, corn, nut, oil poppy, evening primrose, barley, pumpkin seeds, grape seeds, peas, sesame, soya, or sunflower.

6. A composition according to claim 1, wherein the concentration of iturinic lipopeptides is between 20 and 50 g/L and the surfactants are surfactin, the concentration of the surfactin in the composition being between 1 and 40 g/L.

7. A composition according to claim 1, wherein the concentration of iturinic lipopeptides is between 20 and 50 g/L and the surfactants are fengycin, the concentration of fengycin in the composition being between 1 and 100 g/L.

8. A composition according to claim 1, wherein the concentration of iturinic lipopeptides is between 20 and 100 g/L and the surfactants are surfactin and fengycin, the concentration of surfactin is between 1 and 40 g/L and the concentration of fengycin is between 1 and 100 g/L.

9. A composition according to claim 1, wherein the concentration of iturinic lipopeptides is between 20 and 100 g/L and the surfactants are a vegetable oil extract, polyglycoside derivatives and surfactin.

10. A composition according to claim 1, wherein the concentration of iturinic lipopeptides is between 20 and 100 g/L and the surfactants are an extract of methylated vegetable seed oil and coconut oil polyglycerol ester.

11. A method for preparing a composition g/L, and one or more of iturinic lipopeptides above 20 g/L, and one or more surfactant(s) chosen from the families of: anionic surfactants, non-ionic surfactants or oils, the method comprising preparing a solution with a high content of the iturinic lipopeptides and adding the surfactants.

12. A method according to claim 11, further comprising a dehydration step.

13. A composition according to claim 1, wherein the composition is an agri-food, phytosanitary or cosmetic composition.

14. A composition comprising a concentration of iturinic lipopeptides above 20 g/L, and one or more surfactant(s) chosen from the families of: anionic surfactants, non-ionic surfactants or oils, the composition being a medical or pharmaceutical composition.
